(19) United States
(12) Patent Application Publication
(10) Pub. No.: US 2005/0020685 A1
(43) Pub. Date: Jan. 27, 2005

(54) Process for recovery of 6-aminopenicillanic acid from an aqueous discharge stream

(76) Inventors: Paul M. Skonezny, Baldwinsville, NY (US); Robert J. Stankavage, Bridgeport, NY (US); Gregory L. Stein, Mallory, NY (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/865,422

(22) Filed: Jun. 10, 2004

Related U.S. Application Data
(60) Provisional application No. 60/477,862, filed on Jun. 12, 2003.

(51) Int. Cl. A61K 31/198; C07C 323/25

(52) U.S. Cl. 514/562; 562/558

(57) ABSTRACT

A process for recovering residual amounts of 6-APA, on the order of 10 g/L or less, from a predominantly aqueous liquor containing phenoxyacetic acid and less than about 2% organic solvents.

This application claims a benefit of priority from U.S. Provisional Application No. 60/477,862, the entire disclosure of which is herein incorporated by reference.

### TECHNICAL BACKGROUND AND FIELD OF INDUSTRIAL APPLICABILITY

This application relates to a new and useful process for production of 6-aminopenicillanic acid (6-APA) in which the total recovery of 6-APA is improved by the recovery of residual 6-APA from the discharge stream generated after initial recovery of that product. The 6-APA is advantageously derived from the reactive conversion of a penicillin starting material. In particular, the invention relates to a process of recovering and re-using residual 6-APA from a predominantly aqueous discharge stream.

### BACKGROUND OF THE INVENTION

6-APA is an important commercial intermediate in the production of β-lactam antibiotics such as ampicillin and amoxicillin. It can be prepared, for example, by the enzymatic conversion of penicillin V to 6-APA, as described for example by Mollgaard and Karlsen (Ann. of the New York Acad. Sci. (1988), 360-365). The product is obtained by crystallization from an aqueous solution, where a significant amount of yield can be lost due to the relatively high solubility of 6-APA in water.

6-APA can also be prepared by reactive conversion of other penicillins as well, with the formation of various chemical species as by-products. Japanese Patent Abstract JP 59082097 teaches ultrafiltration of a crude aqueous solution of benzyl penicillin (penicillin G) with a membrane having a cut-off weight of ≧5,000, and treating the product with immobilized penicillin acylase.

Several approaches have been evaluated for the recovery of 6-APA from solution. These have included the use of solutions in phase separation recovery, as well as physical separation techniques such as reverse osmosis or resin separation.

U.S. Pat. No. 3,008,956 describes a method for recovering 6-APA from dilute aqueous solutions by reactive extraction using an excess of a secondary amine reagent and organic solvents. The 6-APA is removed from the aqueous phase. This method however has proven to be impractical because the costs of using such reagents and solvents outweigh any benefit in increased yield.

PCT World Patent Application WO99/48895 and European Patent Application EP 0950660 also disclose a process for recovering 6-APA from a mother liquor in which it is formed by enzymatic conversion of penicillin G. The mother liquor is contacted with a recovery solution that is a solution of one or more amines in approximately equivalent parts by volume (0.5 to 2) in relation to the mother liquor. The 6-APA is separated into the organic amine phase, then further extracted into an acidified aqueous phase, from which it is crystallized. The initial extraction into the amine phase is accomplished using a countercurrent extraction or centrifugal extraction. Although the costs of this process might be reduced in comparison to that described by U.S. Pat. No. 3,008,956, there would still be significant costs incurred by the use and necessary recovery of the expensive secondary amines and solvents.

Shengwu Gongchen Xeubao, 14 (4), 468-471 (1998) describes the use of a reverse osmosis system to concentrate 6-APA from a dilute mother liquor, however this process involves a large expenditure of energy to reduce the volume 20-30×, as well as a significant investment for capital equipment.

The use of anionic and cationic resins as means for absorption of 6-APA from aqueous solutions has also been described in the art. U.S. Pat. No. 6,110,699 describes using a series of resins, a first nonionic resin followed by multiple anionic resins of strong or weak character, to separate 6-APA and phenylacetic acid, a by-product, from a solution obtained by enzymatic conversion of penicillin G. The patent teaches that preferably the 6-APA is absorbed onto the anionic resins and eluted therefrom using salt solutions or organic solvents.

The use of nonionic resins, as discussed above, where it has been practiced in the art has been accompanied by either functionalization of the resin or use of acidic or alkaline eluting agents to remove the absorbed component. It has not been reported, nor has it previously been known in the art to use nonionic resins to recover residual amounts of 6-APA from an aqueous discharge stream or mother liquor using water as an eluting agent.

It is therefore an objective of the present invention to provide a process for recovery of residual 6-APA from an aqueous solution, in particular a predominantly aqueous solution, using one or more nonionic resins in a method that does not require the use of acids, alkalis or solvents as eluting agents.

It is a further object of the invention to provide a process in which by-products of a reactive conversion of penicillin V to 6-APA, in particular phenoxyacetic acid (POAc) and inorganic salts are selectively separated from the residual 6-APA by a resin separation process using nonionic resins.

Another objective of the present invention is to provide an improved method for preparation of 6-APA by reactive conversion of penicillin V, in which the overall recovery of 6-APA from such a process is maximized by recovery of residual product from the process waste streams. The residual product can then be used as input to subsequent conversions.

These and other objectives are met by the various embodiments of the present invention.

### SUMMARY OF THE INVENTION

In one aspect, the invention comprises a process for the production of 6-aminopenicillanic acid: 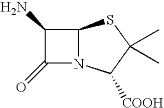
that comprises (a) performing a primary recovery of 6-aminopenicillanic acid from a mixture formed by reactive conversion of penicillin V, which generates a discharge stream containing residual 6-aminopenicillanic acid and phenoxyacetic acid; (b) recovering an aqueous 6-aminopenicillanic acid stream from the discharge stream; and (c) optionally, recycling the recovered 6-aminopenicillanic acid stream into a subsequent reactive conversion. Preferably, the process is one in which the Penicillin V is generated from a microbial fermentation, however the recovery of the residual 6-APA may also be implemented in other processes in which Penicillin V is obtained by other means, for example synthetic or other semisynthetic means. More particularly, the process comprises recovering residual 6-aminopenicillanic acid from the discharge stream by adjusting the pH of the discharge stream to a value of from about 3.3 to about 3.5. The discharge stream is then contacted with each of one or more nonionic resins, and eluting concentrated residual 6-aminopenicillanic acid from each of said resins with water.

The process of the invention may comprise a part of any process in which a penicillin starting material such as Penicillin V is converted to 6-APA, irrespective of its source, and should not be interpreted as limited in any way with respect to the origination of this starting material. Rather, the penicillin starting material may be obtained by any means known in the art, including microbial fermentation, semi-synthetic or synthetic preparation methods. In a preferred embodiment, the process is utilized to improve recovery of 6-APA in a process of producing 6-APA from Penicillin V comprising: forming a solution of Pen V acid in water; performing a reactive conversion of the Pen V acid using an enzyme, to form a mixture comprising 6-APA and phenoxyacetic acid; performing primary recovery of 6-APA from this mixture, thereby obtaining a discharge stream comprising residual 6-APA; adjusting the pH of the discharge stream to a value of from about 3.3 to about 3.5; contacting the discharge stream with one or more nonionic resins; and eluting 6-APA from the one or more nonionic resins with water.

The invention in its various embodiments provides an advantage in that it uses no organic solvents or salt solutions to recover the 6-APA and the rich fractions formed by elution from the nonionic resins are suitable for recycle ‘as is’ in the reactive conversion of Penicillin V.

### BRIEF DESCRIPTION OF THE DRAWINGS

FIGURE 1 is a schematic flow diagram showing a representative process for the preparation of 6-APA from Penicillin V.

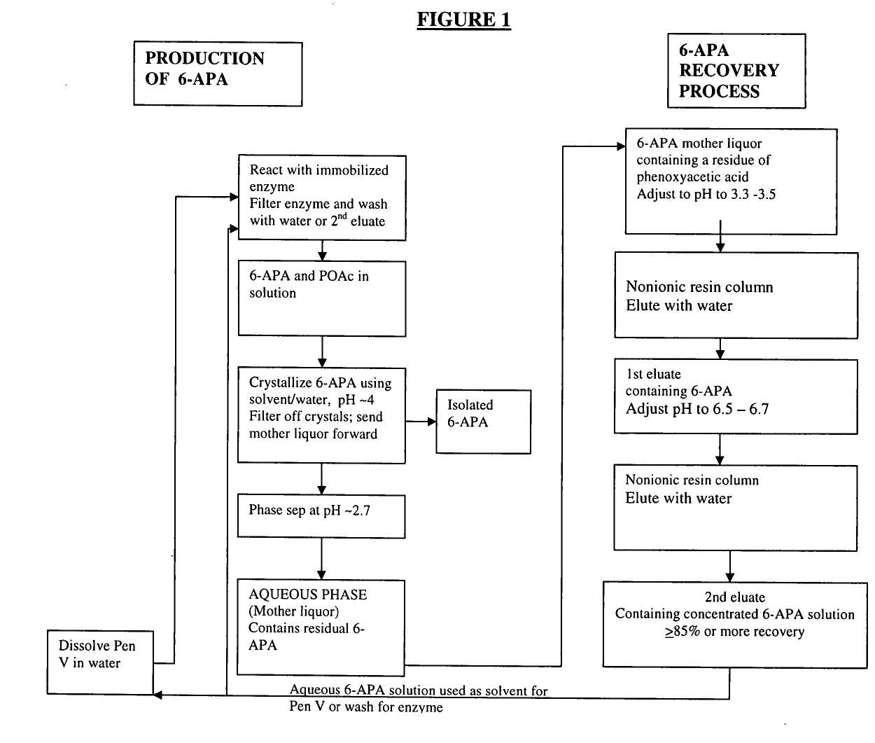

### DETAILED DESCRIPTION OF PREFERRED EMBODIMENTS OF THE INVENTION

According to the invention, a process of producing 6-APA from a penicillin source, preferably penicillin V acid, is improved by the enhanced recovery of the 6-APA product from discharge streams that previously would have been discarded, thereby causing loss of product ranging up to about 5-10% weight. Penicillin V acid is also referred to herein as penicillin V or pen V. The recovery process according to the invention is applied to predominantly aqueous solutions, i.e. solutions containing less than about 2% by volume of organic solvents, in which the amount or residual 6-APA is from about 3 g/L to about 7 g/L.

Crystallized penicillin V can be dissolved in water and subsequently converted to 6-APA by reactive conversion. In a preferred embodiment of this invention, the penicillin V is enzymatically de-acylated at the 6-position to form 6-APA and phenoxyacetic acid (POAc) as a by-product:

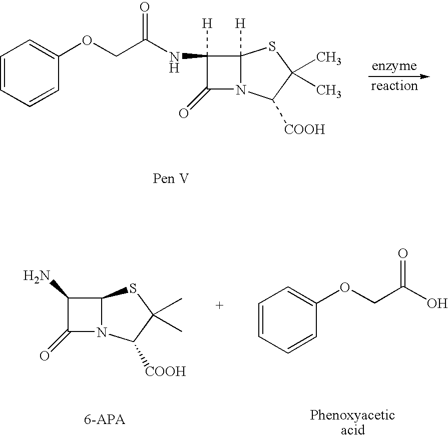

The enzymatic conversion may be carried out using an enzyme selected from the group consisting of amidases and acylases. In a preferred embodiment, an amidase enzyme, such as one derived from Fusarium oxysporum is used. Preferably the enzyme is immobilized. Suitable amidase enzymes are described in co-pending, commonly assigned EP Application No. 718402 and U.S. Pat. No. 5,516,679; the entire subject matter of each is herein incorporated by reference in its entirety. When the starting material is penicillin V, the enzymatic deacylation produces phenoxyacetic acid (POAc) as a side chain by-product, which remains in aqueous solution with the 6-APA. It is possible that the recovery process for residual 6-APA according to the invention could also be applied to processes in which the starting material is another penicillin or beta-lactam compound, and accordingly such applications should also be considered within the broad scope of the invention; however if a molecule other than penicillin V is used, it would be expected that the side chain by-product would be a different chemical entity that could predictably be formed by the splitting action of the selected enzyme.

After the splitting reaction, the enzyme is filtered off and washed, and the aqueous stream is combined with a solvent such as butyl acetate and the pH lowered to the point at which the zwitterionic form of 6-APA precipitates, preferably about 3.9. After filtration and washing, the discharge stream or mother liquor contains less than about 10%, preferably about 5 to 9% of the total 6-APA formed by the enzymatic conversion, and all of the POAc. The pH of the mother liquor is adjusted to a pH of from about 2.3 to about 3.3, preferably pH 2.7, where >95% of the POAc is recovered in the butyl acetate phase. The 6-APA is then recovered from the aqueous phase by passing the solution through a small column of nonionic resin to remove color, residual solvent, and residual POAc. The solution is then adjusted to a pH between about 6.5 to 6.7, preferably about pH 6.5. The stream is then passed through a larger nonionic resin column to allow the salts and early eluting peaks to be separated from 6-APA. The resulting solution is then used as the water to dissolve penicillin V acid for a subsequent splitting or as wash for the enzyme. The resins can be regenerated as necessary by passing a NaOH solution through the resin column followed by a water wash.

The nonionic resins used in the process of the invention are suitably any nonfunctionalized nonionic resins having the binding capacity to separate 6-APA from the residual POAc and inorganic salts. Exemplary resins are styrene divinylbenzene copolymer resins such as Amberlite XAD 16, XAD 1600 and XAD 1180, which are manufactured by Rohm and Haas Co. The invention provides a significant advantage in that the elution from the resin columns is accomplished using only water as the eluting agent, unlike other processes described in the prior art, in which solvents, salts, acid and alkalis are used for that purpose.

Where pH adjustment is required throughout the process, it may be accomplished using standard materials known in the art for that purpose, for example sulfuric acid and ammonium hydroxide, among others.

The residual 6-APA recovered by the process of the invention is usually in the amount of less than about 10 g/L based on the total volume of the original discharge stream. Practice of the invention improves the overall yield in a continuous process of preparing 6-APA from a penicillin starting material by recovering a residual amount of 6-APA from each run and recycling it into subsequent runs. By applying a resin purification and concentration process to the waste stream containing this residual product, and subsequently re-using this waste stream without further processing, the invention provides an increase in the overall yield of such a process. The 6-APA produced according to the processes described herein may be used as a starting reactant for the preparation of various semisynthetic penicillins, such as ampicillin and amoxicillin.

The invention will be further described according to the following examples, which are not intended to be limiting in any way.

### EXAMPLES

### Example 1

Preparation of a Solution of 6-APA

A solution of Penicillin V in water at pH 8.2 was treated with immobilized pen V amidase enzyme. The pH was maintained at 8.2 by addition of ammonium hydroxide until the penicillin V was converted to 6-APA. The resulting solution of 6-APA (7-8% w/v) was then filtered to remove the enzyme. The enzyme was washed with water, and the combined 6-APA solution and wash was carbon treated to remove color. The carbon was removed by filtration and washed with water. The 6-APA filtrate and wash were combined for the crystallization step.

### Example 2

Crystallization of 6-APA

At 20-25° C., one-half volume of butyl acetate was added to the aqueous 6-APA solution formed, for example, according to Example 2 and with vigorous stirring, the pH was slowly reduced to pH 3.9 with concentrated sulfuric acid. The resulting crystal slurry was maintained at pH 3.9 for 20 minutes. The crystals were then collected on a filter and washed with water and butyl acetate, then dried at 45-50° C. in vacuo. The loss in the aqueous mother liquor was ˜3-4 g/L (˜5-6% yield).

### Example 3

Mother Liquor Pre-Processing

The pH of the two-phase mother liquor from Example 2 was lowered to pH 2.7 with concentrated sulfuric acid. The butyl acetate layer was separated off and the aqueous layer was retained containing ˜3-4 g/L 6-APA. This was used as the input to the mother liquor recovery process.

### Example 4

Recovery of 6-APA by Resin Columns

The aqueous 6-APA solution from Example 3 was adjusted to pH 3.3-3.5 with concentrated ammonium hydroxide and then passed through a column (0.125V; h:d ratio=4.6) of Amberlite XAD 1600 resin at a rate of 0.20-0.24 column volumes per minute. This was followed with 0.25 volume water. This step removed residual phenoxyacetic acid, color bodies, residual solvent and late eluting impurities. The column was regenerated by passing 0.5 volume 15% NaOH and 10 bed volumes of water.

The fraction containing the 6-APA (90-95% recovery) was at pH 4.4-4.8 and was then adjusted to pH 6.5 to 6.7 with concentrated ammonium hydroxide. This was passed through a second column (0.5V; h:d ratio=15) of XAD1600 resin at a rate of 0.09-0.10 column volumes per minute. This was followed with 1.2 volumes of water. This step removed salts, any remaining residual solvent and early eluting impurities. Regeneration of this column when necessary was accomplished by passing 0.13 bed volumes of 15% NaOH and 5 volumes of water through the column.

The fraction containing 6-APA (˜0.75 of the original volume; 93-96% recovery) was suitable for use, as is, as the solvent to dissolve Penicillin V and enzyme wash following the preparation of 6-APA.

### Example 5

Preparation and Isolation of 6-APA Using Recovered Mother Liquor in Place of Water

The procedure of Example 1 was followed using the recovered 6-APA solution from Example 3 in place of water. The 6-APA solution resulting from this processing was then crystallized by the procedure of Example 2.

A table comparing the results using multiple recovery cycles and 6-APA isolations is listed below:

| | Recovered | | | | |
|---|---|---|---|---|---|
| | g 6-APA | % 6-APA | | % Weight | |
| | Input to | recovered | g 6APA | Yield | |
| 6-APA Run | cycle | From ML | output | Increase | Absorbance |
| RUN 1 no | 0 | 87.8 | 27.81 | N/A | 0.030 |
| ML Rec | | | | | |
| cycle 1 with | 1.389 | 80.1 | 28.85 | 4.2 | 0.029 |
| ML rec | | | | | |
| cycle 2 with | 1.310 | 83.6 | 29.20 | 5.4 | 0.031 |
| ML rec | | | | | |
| cycle 3 with | 1.172 | 84.3 | 28.50 | 2.9 | 0.023 |
| ML rec | | | | | |
| cycle 4 with | 1.241 | 86.9 | 29.29 | 5.8 | 0.026 |
| ML rec | | | | | |
| cycle 5 with | 1.174 | 91.5 | 29.06 | 4.9 | 0.034 |
| ML rec | | | | | |
| cycle 6 with | 1.168 | 88.6 | 28.92 | 4.4 | 0.028 |
| ML rec | | | | | |
| cycle 7 with | 1.426 | 88.8 | 29.03 | 4.8 | 0.029 |
| ML rec | | | | | |
| cycle 8 with | 1.271 | 88.2 | 29.27 | 5.7 | 0.028 |
| ML rec | | | | | |
| Control no | 0 | | 27.58 | N/A | 0.023 |
| ML rec | | | | | |

ML rec = Mother Liquor recovery
$\begin{matrix}\% Weight \\ Yield Increase\end{matrix} = \frac{g 6 \text{-} APA output}{\sum ( Run 1 + Control Run 6 \text{-} APA output ) / 2}$

Run 1 and the Control run contained no 6-APA recovered from mother liquor. An additional average 4.8% weight yield of 6-APA, in comparison to the runs performed without mother liquor recovery, was obtained by recovering the 6-APA from the mother liquor and adding it to the subsequent cycle versus no mother liquor recovery runs. The absorbance of the 6-APA, which is an indicator of quality, showed no increase throughout the series indicating that the recovery process produced 6-APA with equivalent absorbances to the runs where no mother liquor recovery was performed.
Method to Determine Absorbance of a Solution of 6-APA

1.0 g of 6-APA is dissolved in 10 mL of 10% (w/v) Na₂CO₃ in water by shaking. The solution is filtered over S&S No.576 filter paper and the absorbance of the filtrate is measured in a spectrophotometer at 430 nm in a 1 cm cell (vs. 10% Na₂CO₃ in water).

While the foregoing examples describe various embodiments of the present invention, other and further embodiments, modifications, and improvements will be known to those skilled in the art, and it is intended to include all such further embodiments, modifications, and improvements and come within the true scope of the claims as set forth below.

1. A process for the production of 6-aminopenicillanic acid comprising a primary recovery of 6-aminopenicillanic acid from a mixture formed by reactive conversion of penicillin V, thereby generating a discharge stream containing residual 6-aminopenicillanic acid and phenoxyacetic acid; and recovering the residual 6-aminopenicillanic acid from the discharge stream.

2. The process of claim 1 wherein the residual 6-aminopenicillanic acid is recovered from the discharge stream by adjusting the pH of the discharge stream to a value of from about 3.3 to about 3.5, then contacting said discharge stream with each of one or more nonionic resins, and eluting residual 6-aminopenicillanic acid from each of said resins with water.

3. The process of claim 2 comprising contacting the discharge stream with a first nonionic resin and eluting residual 6-APA from said first nonionic resin with water to form a first rich fraction, and subsequently adjusting the pH of said first fraction to 6.5 to 6.7, contacting said first rich fraction with a second nonionic resin and eluting 6-APA from said second nonionic resin with water to form a second rich fraction.

4. The process of claim 3 further comprising re-using the first rich fraction or the second rich fraction in a process for reactive conversion of Penicillin V to 6-APA.

5. The process of claim 2 wherein the one or more resins is selected from the group consisting of styrene divinylbenzene copolymer resins.

6. The process of claim 5 wherein the one or more resins is selected from the styrene divinylbenzene copolymer resins XAD 16, XAD1180 or XAD1600.

7. The process of claim 2 wherein the discharge stream comprises less than about 10 grams per liter of 6-APA in a predominantly aqueous solution.

8. The process of claim 7 wherein the discharge stream comprises less than about 5 grams per liter of 6-APA in predominantly aqueous solution.

9. The process of claim 1 wherein the penicillin V is derived from a microbial fermentation process, and the reactive conversion to 6-APA is performed using an enzyme.

10. The process of claim 9 wherein the enzyme is an amidase.

11. A process for the improved recovery of 6-APA in a process of producing 6-APA from Penicillin V acid comprising:
a) dissolving Penicillin V acid solids in water to form an aqueous solution;
b) performing a reactive conversion of the Penicillin V acid in aqueous solution using an enzyme, to form a mixture comprising 6-APA and phenoxyacetic acid;
c) performing primary recovery of 6-APA from the mixture of step (b), thereby obtaining a discharge stream comprising residual 6-APA;
d) adjusting the pH of the discharge stream to a value of from about 3.3 to about 3.5;
e) contacting the discharge stream of step (d) with one or more nonionic resins;
f) eluting 6-APA from the one or more nonionic resins with water; and
g) optionally, recycling the recovered 6-APA stream into a subsequent reactive conversion.

12. The process of claim 2 wherein the one or more resins comprise a first nonionic resin in a column and a second nonionic resin in a column.

13. A process to recover 6-aminopenicillanic acid (6-APA) from a predominantly aqueous discharge stream comprising less than about 2% organic solvent, wherein the 6-APA is passed through one or more nonionic resins using only water as an eluting agent to produce rich fractions comprising 6-APA, collecting the rich fractions of 6-APA, and reusing said rich fractions in a process of making 6-APA.

14. The process according to claim 13 wherein the rich fractions comprising 6-APA are used as the solvent for the dissolution of penicillin V to be used in an enzymatic preparation of 6-APA.

15. The process according to claim 13 wherein the rich fractions are used to wash the enzyme used in enzymatic preparation of 6-APA.

16. The process of claim 2 further comprising regeneration of one or more of the nonionic resins.
